(19) United States
(12) Patent Application Publication
(10) Pub. No.: US 2022/0160739 A1
(43) Pub. Date: May 26, 2022

(54) METHODS FOR TREATING SUBJECTS WITH CHRONIC KIDNEY DISEASE

(71) Applicant: Janssen Pharmaceutica NV, Beerse (BE)

(72) Inventor: Norman R. Rosenthal, Hopewell, NJ (US)

(21) Appl. No.: 17/440,990

(22) PCT Filed: Mar. 25, 2020
(86) PCT No.: PCT/EP2020/058415

§ 371 Date: Sep. 20, 2021

Related U.S. Application Data
(60) Provisional application No. 62/835,550, filed on Apr. 18, 2019.
(60) Provisional application No. 62/823,724, filed on Mar. 26, 2019.
(60) Provisional application No. 62/823,722, filed on Mar. 26, 2019.
(60) Provisional application No. 62/823,719, filed on Mar. 26, 2019.

(51) Int. Cl. A61K 31/7042; A61K 45/06; A61P 13/12; A61P 3/10

(52) U.S. Cl. CPC A61K 31/7042; A61P 3/10; A61P 13/12; A61K 45/06

(57) ABSTRACT

The present disclosure provides methods for treating chronic kidney disease, comprising administering to a patient in need thereof, a therapeutically effective amount of canagliflozin; wherein the patient is diagnosed with Type II diabetes mellitus.

### CROSS REFERENCE TO RELATED APPLICATIONS

This application claims priority to U.S. Provisional Patent Application No. 62/823,719, filed Mar. 26, 2019, United States Provisional Patent Application No. 62/823,722, filed Mar. 26, 2019, U.S. Provisional Patent Application No. 62/823,724, filed Mar. 26, 2019, U.S. Provisional Patent Application No. 62/835,550, filed Apr. 18, 2019, the disclosures of which is hereby incorporated by reference in their entireties.

### TECHNICAL FIELD

This invention relates to methods for treating subjects with chronic kidney disease.

### BACKGROUND

Subjects with type 2 diabetes and chronic kidney disease (CKD) are at high risk of developing end-stage kidney disease (ESKD) and cardiovascular events, and of having a reduced life expectancy, despite the use of current standard of care. Once patients with diabetic kidney disease develop end-stage kidney disease, these individuals have a reduced life expectancy, with a 5-year survival rate for patients on dialysis in the United States of 36% and an even lower percentage in developing countries. Old age and long duration and high number of comorbidities are typical of a patient population having established CKD, a population that has a high unmet medical need.

Kidneys are bean-shaped organs, located near the middle of the back. Inside each kidney about a million tiny structures called nephrons filter blood. They remove waste products and extra water, which become urine. Damage to the nephrons represents an important form of kidney disease. This damage may leave kidneys unable to remove wastes. Some damage, e.g., damage related to hyperfiltration can occur slowly over years, initially often without obvious symptoms.

At the single-nephron level, hyperfiltration is hypothesized to be an early link in the chain of events that lead from intraglomerular hypertension to albuminuria and, subsequently, to reduced Glomerular Filtration Rate (GFR). Based on this, hyperfiltration therefore represents a risk for subsequent renal injury and could be classified as an early manifestation of renal pathology often referred to as the hyperfiltrative stage. Such renal hyperfiltration can lead to early glomerular lesions and to microalbuminuria, which itself can lead to macroalbuminuria and to end-stage renal disease.

Creatinine is a breakdown product of creatine phosphate in muscle tissue, and is usually produced at a constant rate in the body. Serum creatinine is an important indicator of renal health, because it is an easily measured byproduct of muscle metabolism that is excreted unchanged by the kidneys. Creatinine is removed from the blood chiefly by the kidneys, primarily by glomerular filtration, but also by proximal tubular secretion. Little or no tubular reabsorption of creatinine occurs. If the filtration in the kidney is deficient, creatinine blood levels rise. Therefore, creatinine levels in blood and urine may be used to calculate the creatinine clearance (CrCl), which correlates with the glomerular filtration rate (GFR). Blood creatinine levels may also be used alone to estimate the GFR (eGFR).

Albuminuria is a condition where albumin is present in the urine. In healthy individuals, albumin is filtered by the kidneys. When the kidneys do not properly filter large molecules (such as albumin) from the urine, albumin is excreted in urine and is typically a sign of kidney damage or excessive salt intake. Albuminuria can also occur in patients with long-standing diabetes mellitus, either Type I (1) or Type II (2) diabetes mellitus. Urine albumin may be measured by dipstick or as direct measure of the amount of protein excreted in total volume of urine collected over a 24 hour period.

Diabetic nephropathy is one of the microvascular complications of diabetes mellitus and is characterized by persistent albuminuria and a progressive decline in renal function. Hyperglycemia is an important contributor to the onset and progression of diabetic nephropathy.

The clinical progression of diabetic nephropathy in patients with T1DM (Type 1 Diabetes Mellitus) is well characterized. Initially, hyperfiltration accompanied by increases in glomerular filtration rate (GFR) and increased renal plasma flow is seen. A meta-analysis found that the presence of hyperfiltration in patients with T1DM more than doubled the risk of developing micro- or macroalbuminuria. This phase is followed by reductions in GFR and the development of microalbuminuria, defined as urinary albumin excretion of ≥30 mg/day (or 20 μg/min) and <300 mg/24 h (or <200 μg/min), which may be accompanied by increases in blood pressure. Later in the progression of the disease as GFR continues to decline, overt proteinuria (i.e., macroalbuminuria), defined as urinary albumin excretion of ≥300 mg/day ensues and is associated with worsening hypertension. Eventually, ESKD (End Stage Kidney Disease) progresses, leading to the need for renal replacement therapy.

In patients with Type 2 Diabetes Mellitus (T2DM), the clinical progression is variable, primarily due to multiple renal insults, including not only hyperglycemia, but also vascular pathology resulting in ischemic renal injury. However, other common features are likely to contribute to renal injury in patients with T2DM and include hyperfiltration at the level of the single nephron, proximal tubular glucotoxicity, and a stimulus for tubular cell growth as a result of enhanced sodium coupled glucose transport into tubular cells.

The magnitude of albuminuria positively correlates with the development of ESKD and adverse CV outcomes. Treatment-related reductions in albuminuria in patients with T2DM and albuminuria using agents acting by a hemodynamic mechanism (i.e., ACEi and ARBs) are correlated with reductions in the progression of diabetic nephropathy and in the incidence of adverse CV outcomes. Thus, agents acting by a unique hemodynamic mechanism to reduce albuminuria beyond that seen with other antihypertensive or antihyperglycemic agents and which are additive to agents disrupting the renin-angiotensin system may exert reno-protective effects and possibly reduce adverse CV outcomes in diabetic nephropathy.

Despite the current standard of care, subjects with Type II diabetes mellitus and chronic kidney disease are at a high risk of developing ESKD and cardiovascular events, and of having a reduced life expectancy. What is needed are methods of treating patients in more advanced stages of chronic kidney disease.

### SUMMARY

In some aspects, the disclosure is directed to treating patients with Type II diabetes mellitus and chronic kidney disease (CKD). In particular embodiments, the patients have stage 2-3 kidney disease. In other embodiments, the patients also have macroalbuminuria. The methods comprise administering a therapeutically effective amount of canagliflozin. In other embodiments, the methods further comprise a concomitant standard of care comprising administering an angiotensin-converting enzyme inhibitor and/or angiotensin receptor blocker. The methods described herein have also been proven to be clinically safe and/or clinically effective.

In other aspects, the disclosure is directed to methods of treating a diabetic patient with chronic kidney disease, comprising (a) determining whether the patient has chronic kidney disease; and (b) administering canagliflozin to the patient in a therapeutically effective amount to treat the chronic kidney disease.

In further aspects, the disclosure is directed to methods of selling a drug product comprising canagliflozin, the method comprising selling the drug product, wherein a drug product label for a reference listed drug for the drug product includes instructions for treating chronic kidney disease.

In yet other aspects, the disclosure is directed to pharmaceutical products comprising a clinically proven safe and clinically proven effective amount of canagliflozin.

### BRIEF DESCRIPTION OF THE DRAWINGS

FIG. 1 is a Kaplan-Meier Plot of the First Occurrence of the Primary Composite Endpoint for Example 1.

FIG. 2 is a line graph of the LS mean change from baseline in HbA_{1c} over time for screening eGFR stratum ≥30 to <45 mL/min/1.73 m².

FIG. 3 is a line graph of the LS mean change from baseline in HbA_{1c} over time for screening eGFR Stratum ≥45 to <60 mL/min/1.73 m².

FIG. 4 is a line graph of the LS mean change from baseline in HbA_{1c} over time for screening eGFR stratum ≥60 to <90 mL/min/1.73 m².

### DETAILED DESCRIPTION OF ILLUSTRATIVE EMBODIMENTS

In the present disclosure the singular forms “a”, “an,” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

When a value is expressed as an approximation by use of the descriptor “about” or “substantially” it will be understood that the particular value forms another embodiment. In general, use of the term “about” or “substantially” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about” or “substantially”. In other cases, the gradations used in a series of values may be used to determine the intended range available to the term “about” or “substantially” for each value. Where present, all ranges are inclusive and combinable. That is, references to values stated in ranges include every value within that range.

When a list is presented, unless stated otherwise, it is to be understood that each individual element of that list and every combination of that list is to be interpreted as a separate embodiment. For example, a list of embodiments presented as “A, B, or C” is to be interpreted as including the embodiments, “A,” “B,” “C,” “A or B,” “A or C,” “B or C,” or “A, B, or C.”

It is to be appreciated that certain features of the invention which are, for clarity, described herein in the context of separate embodiments, may also be provided in combination in a single embodiment. That is, unless obviously incompatible or excluded, each individual embodiment is deemed to be combinable with any other embodiments and such a combination is considered to be another embodiment. Conversely, various features of the invention that are, for brevity, described in the context of a single embodiment, may also be provided separately or in any sub-combination. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Finally, while an embodiment may be described as part of a series of steps or part of a more general structure, each said step may also be considered an independent embodiment in itself

Methods

The present disclosure provides methods for treating chronic kidney disease (CKD), comprising administering to a patient in need thereof, a therapeutically effective amount of canagliflozin. In some aspects, the patient is a diabetic patient. In further aspects, the patient is a diabetic patient with chronic kidney disease. In other aspects, the patient is diagnosed with Type II diabetes mellitus. In other aspects, the patient is further diagnosed with macroalbuminuria.

The methods described herein reflect the effectiveness of canagliflozin in treating a specific subpopulation of patients, i.e., those having Type II diabetes mellitus (T2DM) and chronic kidney disease (CKD). The methods disclosed herein result in benefits to renal outcomes (by any mechanism) in these patients since ACE inhibitors (ACEi) and angiotensin receptor blockers (ARB) became the standard of care for the prevention of the progression of diabetic kidney disease over 15 years ago. The methods described herein reduced the risk of certain primary endpoints by about 25% on top of current standard of care that includes ACEi or ARB therapy. The inventors also found a significant duration of effect and/or reduction in co-morbidity, and that the effects provided by canagliflozin were maintained even after 1 year of treatment. This duration of effects was consistent across all subgroups of tested patients, thereby suggesting a long-lasting treatment option for afflicted patients. Significantly, the finding of benefit for primary endpoints was not driven exclusively by a laboratory component (i.e., doubling of serum creatinine) but was evident by a significant reduction in the ‘hard endpoint’ of ESKD (comprised of adjudicated sustained eGFR <15 mL/min/1.73 m², chronic dialysis or renal transplant).

As used herein, the term “diabetes” includes Types 1 and 2. In some embodiments, diabetes refers to Type 1 diabetes. In other embodiments, diabetes refers to Type 2 diabetes. Diabetes Types 1 and 2 are understood to those skilled in the art. In patients having Type 1 diabetes, the patient's immune system attacks and destroys beta cells in the pancreas that produce insulin. Thus, Type 1 diabetics do not produce insulin. In patients having Type 2 diabetes, the patient's body does not use insulin efficiently. Thus, Type 2 diabetics do not respond to the insulin produced in the body.

The terms “Type 2 diabetes” and “Type II diabetes mellitus” are interchangeable and defined as the condition in which a patient has a fasting (i.e., no caloric intake for about 8 hours) blood glucose or serum glucose concentration greater than about 125 mg/dL (about 6.94 mmol/L), when measured at minimum two independent occasions. Type 2 diabetes is also defined as the condition in which a patient has HbA_{1c} equal to, or greater than about 6.5%, a two hour plasma glucose equal to, or greater than about 200 mg/dL (about 11.1 mmol/L) during an oral glucose tolerance test (OGTT) or a random glucose concentration equal to, or greater than about 200 mg/dL (about 11.1 mmol/L) in conjunction with classic symptoms of hyperglycemia or hyperglycemic crisis. In the absence of unequivocal hyperglycemia, as with most diagnostic tests, a test result diagnostic of diabetes should be repeated to rule out laboratory error.

The assessment of HbA_{1c} may be performed using methods certified by the National Glycohemoglobin Standardization Program and standardized or traceable to the Diabetes Control and Complications Trial reference assay. If a OGTT is carried out, the blood sugar level of a diabetic will be in excess of about 200 mg of glucose/dL (about 11.1 mmol/L) of plasma about 2 hours after about 75 g of glucose have been taken on an empty stomach. In a glucose tolerance test, about 75 g of glucose are administered orally to the patient being tested after a minimum of about 8 hours, typically after about 10 to about 12 hours, of fasting and the blood sugar level is recorded immediately before taking the glucose and about 1 and about 2 hours after taking it. In a healthy patient, the blood sugar level before taking the glucose will be between about 60 and about 110 mg/dL of plasma, less than about 200 mg/dL about 1 hour after taking the glucose and less than about 140 mg/dL after about 2 hours. If, after about 2 hours, the value is between about 140 and about 200 mg, this is regarded as abnormal glucose tolerance.

In some aspects, the patient diagnosed with Type II diabetes mellitus has a measured HbA_{1c} as defined herein. The term “HbA₁_{_{c}}” or “hemoglobin A₁_{_{c}}” refer to the product of a non-enzymatic glycation of the hemoglobin B chain and its determination is well known to one skilled in the art. In monitoring the treatment of diabetes mellitus the HbA_{1c} value is of exceptional importance. As its production depends essentially on the blood sugar level and the life of the erythrocytes, the HbA_{1c} in the sense of a “blood sugar memory” reflects the average blood sugar levels of the preceding about 4 to about 6 weeks. In certain embodiments, the patient treated according to the methods described herein has a measured HbA_{1c} in the range of about 7% to about 10.5%, such as in the range of ≥7.0% and ≤10.5%. In other embodiments, the patient has a measured HbA_{1c} of about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.8%, about 8.9%, about 9%, about 9.1%, about 9.2%, about 9.3%, about 9.4%, about 9.5%, about 9.6%, about 9.7%, about 9.8%, about 9.9%, about 10%, about 10.1%, about 10.2%, about 10.3%, about 10.4%, or about 10.5%. In other embodiments, the patient has a measured HbA_{1c} of about 7% to about 10%, about 7% to about 9.5%, about 7% to about 9%, about 7% to about 8.5%, about % to about 8%, about 7.5% to about 10.5%, about 8% to about 10.5%, about 8.5% to about 10.5%, about 9% to about 10.5%, or about 9.5 to about 10.5%.

As disclosed herein, the patients treated have chronic kidney disease. In some embodiments, the patient has stage 2 chronic kidney disease. In other embodiments, the patient has stage 3 chronic kidney disease. In further embodiments, the patient has stage 2-3 chronic kidney disease.

The methods described herein may include a determination that the patient has chronic kidney disease. Typically, that determination is made by an attending physician. A diagnosis or determination of chronic kidney disease may be determined using techniques known by those of skill in the art. In some embodiments, the chronic kidney disease is determined by one or more of a blood test, urine test, kidney imaging, or kidney biopsy. Preferably, the chronic kidney disease is diagnosed by a blood test. More preferably, the blood test measures the estimated glomerular filtration rate.

In some aspects, a patient having chronic kidney disease has an eGFR of about 30 to less than about 90 mL/min/1.73 m², such as an eGFR ≥30 to <90 mL/min/1.73 m². In some embodiments, the patient has an eGFR of about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, or about 89 mL/min/1.73 m². In further embodiments, the patient has an eGFR of about 30 to about 89, about 30 to about 85, about 30 to about 80, about 30 to about 75, about 30 to about 70, about 30 to about 65, about 30 to about 65, about 30 to about 60, about 30 to about 55, about 30 to about 50, about 35 to about 89, about 30 to about 85, about 35 to about 80, about 35 to about 75, about 35 to about 70, about 35 to about 65, about 35 to about 60, about 35 to about 55, about 35 to about 50, about 40 to about 89, about 40 to about 85, about 40 to about 80, about 40 to about 75, about 40 to about 70, about 40 to about 65, about 40 to about 60, about 40 to about 55, about 40 to about 50, about 40 to about 45, about 50 to about 89, about 50 to about 85, about 50 to about 80, about 50 to about 75, about 50 to about 70, about 50 to about 65, about 50 to about 60, about 50 to about 55, about 55 to about 89, about 55 to about 85, about 55 to about 80, about 55 to about 75, about 55 to about 70, about 55 to about 65, about 55 to about 60, about 60 to about 89, about 60 to about 85, about 60 to about 80, about 60 to about 75, about 60 to about 70, about 60 to about 65, about 65 to about 89, about 65 to about 85, about 65 to about 80, about 65 to about 75, about 65 to about 70, about 70 to about 89, about 70 to about 85, about 70 to about 80, about 70 to about 75, about 75 to about 89, about 75 to about 85, about 75 to about 80, about 80 to about 89, or about 85 to about 89 mL/min/1.73 m². In other aspects, the patient has an eGFR of about 30 to about 45 mL/min/1.73 m², such as an eGFR ≥30 to <45 mL/min/1.73 m². In further embodiments, the patient has an eGFR of about 30 to about 40, about 30 to about 35, about 35 to about 45, about 35 to about 40, or about 40 to about 45 mL/min/1.73 m². In yet further aspects, the patient has an eGFR of about 45 to about 59 mL/min/1.73 m², such as an eGFR ≥45 to <60 mL/min/1.73 m². In certain embodiments, the eGFR is about 45 to about 59, about 45 to about 55, about 45 to about 50, about 50 to about 59, or about 50 to about 55 mL/min/1.73 m². In still other aspects, the patient has an eGFR of about 60 to about 89 mL/min/1.73 m², such as an eGFR of ≥60 to <90 mL/min/1.73 m². In further embodiments, the eGFR has an eGFR of about 60 to about 85, about 60 to about 80, about 60 to about 75, about 60 to about 70, about 65 to about 89, about 65 to about 85, about 65 to about 80, about 65 to about 75, about 70 to about 89, about 70 to about 85, about 70 to about 80, about 75 to about 89, or about 75 to about 85 mL/min/1.73 m².

In other aspects, the patient has stage 2 chronic kidney disease. As used herein “stage 2 chronic kidney disease” refers to a patient having an eGFR of about 60 to about 89 mL/min/1.73 m², such as ≥60 to <90 mL/min/1.73 m². A patient having stage 2 chronic kidney disease may have no symptoms. To the extent there are signs of kidney damage in these patients, they include, without limitation, protein in the urine, physical damage to one or both kidneys, or any combination thereof. In certain embodiments, patients having stage 2 chronic kidney disease have existing kidney damage.

In other aspects, the patient has stage 3 chronic kidney disease. As used herein “stage 3 chronic kidney disease” refers to a patient having an eGFR of about 30 to about 59 mL/min/1.73 m², such as ≥30 to <60 mL/min/1.73 m². A patient having stage 3 chronic kidney disease may have no symptoms. To the extent there are signs of kidney damage in these patients, they include, without limitation, protein in the urine, physical damage to one or both kidneys, swelling in the hands and/or feet, back pain, urinating more or less than normal, or any combination thereof. In certain embodiments, the stage 3 chronic kidney disease patient has moderate kidney damage. In further embodiments, the stage 3 chronic kidney disease may be stage 3a with an eGFR of about 45 to about 59 mL/min/1.73 m². In yet other embodiments, the stage 3 chronic kidney disease is stage 3b with an eGFR of about 30 to about 44 mL/min/1.73 m².

In further aspects, the patient has stage 2-3 chronic kidney disease. As used herein “stage 2-3 chronic kidney disease” refers to a patient having an eGFR of about 30 to about 89 mL/min/1.73 m², including ≥30 to <90 mL/min/1.73 m². The patient having stage 2-3 chronic kidney disease may have no symptoms. To the extent there are signs of kidney damage in these patients, they include, without limitation, protein in the urine, physical damage to one or both kidneys, swelling in the hands and/or feet, back pain, urinating more or less than normal, or any combination thereof. In certain embodiments, the stage 2-3 chronic kidney disease patient has existing kidney damage that may be moderate.

The term “glomerular filtration rate (GFR)” is defined as the volume of fluid filtered from the renal (kidney) glomerular capillaries into the Bowman's capsule per unit time. It is indicative of overall kidney function. The glomerular filtration rate (GFR) may be calculated by measuring any chemical that has a steady level in the blood, and is freely filtered but neither reabsorbed nor secreted by the kidneys. The rate therefore measured is the quantity of the substance in the urine that originated from a calculable volume of blood. The GFR may be determined by injecting inulin into the plasma. Since inulin is neither reabsorbed nor secreted by the kidney after glomerular filtration, its rate of excretion is directly proportional to the rate of filtration of water and solutes across the glomerular filter. A normal GFR value is about 90 to about 125 mL/min/1.73 m², preferably a GFR of about 100 to about 125 mL/min/1.73 m². Other principles to determine GFR involve measuring ⁵¹Cr-EDTA, [¹²⁵I]iothalamate or iohexyl. The GFR is typically recorded in units of volume per time, e.g., milliliters per minute and the formula below can be used:

$$G F R = \frac{( Urine Concentration \times Urine Volume )}{Plasma Concentration}$$

The “estimated glomerular filtration rate” or “eGFR” as used herein is defined as derived at screening from serum creatinine values based on e.g., the Chronic Kidney Disease Epidemiology Collaboration equation, the Cockcroft-Gault formula or the Modification of Diet in Renal Disease formula, which are all known in the art. Subjects with normal renal function are defined as having an eGFR of about 90 mL/min or greater.

In addition to having chronic kidney disease and Type II diabetes mellitus, the patient may have macroalbuminuria. As used herein, the term “macroalbuminuria” refers to a patient whose albumin/creatinine ratio (ACR) is greater than about 300 mg/g. In some embodiments, a patient with macroalbuminuria has an ACR of about 300 to about 5000 mg/g. In further embodiments, a patient with macroalbuminuria has an ACR of about 300, about 400, about 500, about 750, about 1000, about 1250, about 1500, about 1750, about 2000, about 2250, about 2500, about 2750, about 3000, about 3250, about 3500, about 3750, about 4000, about 4250, about 4500, about 4750, or about 5000 mg/g. In other embodiments, a patient with macroalbuminuria has an ACR of about 300 to about 4000, about 300 to about 3000, about 300 to about 2000, about 300 to about 1000, about 1000 to about 5000, about 1000 to about 4000, about 1000 to about 3000, about 1000 to about 2000, about 2000 to about 5000, about 2000 to about 4000, about 2000 to about 3000, about 3000 to about 5000, about 3000 to about 4000, or about 4000 to about 5000 mg/g.

The methods include administering a therapeutically effective amount of the canagliflozin. The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In some embodiments, the therapeutically effective amount of canagliflozin is about 50 to about 500 mg. In further embodiments, the therapeutically effective amount of canagliflozin is about 50 mg, about 100 mg, about 150 mg, about 200 mg, about 250 mg, about 300 mg, about 350 mg, about 400 mg, about 450 mg, or about 500 mg. In other embodiments, the therapeutically effective amount of canagliflozin is about 50 to about 450 mg, about 50 to about 400 mg, about 50 to about 300 mg, about 50 to about 250 mg, about 50 to about 200 mg, about 50 to about 150 mg, about 50 to about 100 mg, about 100 to about 500 mg, about 100 to about 450 mg, about 100 to about 400 mg, about 100 to about 350 mg, about 100 to about 300 mg, about 100 to about 250 mg, about 100 to about 200 mg, about 150 to about 500 mg, about 150 to about 450 mg, about 150 to about 400 mg, about 150 to about 350 mg, about 150 to about 300 mg, about 150 to about 250 mg, about 200 to about 500 mg, about 200 to about 450 mg, about 200 to about 400 mg, about 200 to about 350 mg, about 200 to about 300 mg, about 250 to about 500 mg, about 300 to about 450 mg, about 300 to about 400 mg, about 350 to about 500 mg, about 350 to about 450 mg, or about 400 to about 500 mg. In yet further embodiments, the therapeutically effective amount is about 100 to about 300 mg. In yet further embodiments, the therapeutically effective amount is about 100 mg.

As used herein, unless otherwise noted, the term “canagliflozin” refers to 1,5-anhydro-1-C-(3-{[5-(4-fluorophenyl)thiophen-2-yl]methyl]}-4-methylphenyl)-glucitol of formula (I).

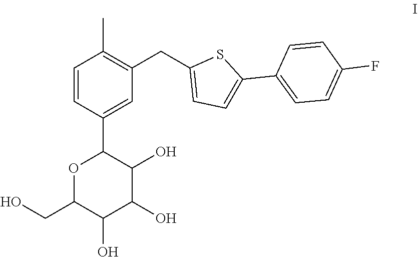 [SMILES: Cc1ccc(C2OC(CO)C(O)C(O)C2O)cc1Cc1ccc(-c2ccc(F)cc2)s1]

In other embodiments, canagliflozin refers to stereoisomers of canagliflozin, such as enantiomers and diastereomers as pure or substantially pure forms. Canagliflozin also refers to racemic mixtures thereof.

In certain embodiments, “canagliflozin” refers to 1,5-anhydro-1-C-(3-{[5-(4-fluorophenyl)thiophen-2-yl]methyl]}-4-methylphenyl)-D-glucitol. In other embodiments, “canagliflozin” refers to a compound that is (1S)-1,5-anhydro-1-C-(3-{[5-(4-fluorophenyl)thiophen-2-yl]methyl]}-4-methylphenyl)-D-glucitol of formula (II):

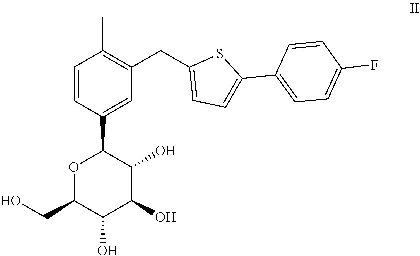 [SMILES: Cc1ccc([C@@H]2O[C@H](CO)[C@@H](O)[C@H](O)[C@H]2O)cc1Cc1ccc(-c2ccc(F)cc2)s1]

As used herein, “canagliflozin” also refers to amorphous or crystalline forms of canagliflozin. In some embodiments, the canagliflozin is a crystalline form. In other embodiments, the canagliflozin is an amorphous form. The crystallinity may be determined by those skilled in the art using one or more techniques such as, e.g., single crystal x-ray diffraction, powder x-ray diffraction, differential scanning calorimetry, melting point, among others.

“Canagliflozin” as used herein includes anhydrous or hydrates thereof. In certain embodiments, the canagliflozin is an anhydrous form. In other embodiments, the canagliflozin is a hydrate thereof. In further embodiments, the canagliflozin hydrate is a hemihydrate thereof. In other embodiments, the canagliflozin is a monohydrate thereof. Thus, canagliflozin, in some embodiments, includes hemihydrates of the compound of formula (I). In other embodiments, canagliflozin includes hemihydrates of the compound of formula (II). In further embodiments, canagliflozin refers to a crystalline, hemihydrate form of the compound of formula (I). In yet further embodiments, canagliflozin refers to a crystalline hemihydrate form of the compound of formula (II). In certain embodiments, canagliflozin refers to the crystalline hemihydrate form of the compound described in International Patent Publication No. WO 2008/069327, the disclosure of which is hereby incorporated by reference in its entirety. In other embodiments, canagliflozin includes monohydrates of the compound of formula (I). In further embodiments, canagliflozin includes monohydrates of the compound of formula (II).

“Canagliflozin” as used herein further refers to solvates thereof. Such solvates include a molecule of a solvent bound through intermolecular forces or chemical bonds to one or more locations of the canagliflozin molecule.

As used herein, “canagliflozin” may also refer to polymorphs thereof. Such polymorphs of canagliflozin include crystalline forms of the molecule, having variations to the crystal lattices of each polymorph.

The term “canagliflozin” may also include pharmaceutically acceptable salts thereof, which may readily be selected by those skilled in the art. A “pharmaceutically acceptable salt” is intended to mean a salt of canagliflozin that is non-toxic, biologically tolerable, or otherwise biologically suitable for administration to the subject. See, e.g., Berge, “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002, which are incorporated herein by reference. Examples of pharmaceutically acceptable salts are those that are pharmacologically effective and suitable for administration to patients without undue toxicity, irritation, or allergic response. Examples of pharmaceutically acceptable salts include sulfates, pyrosulfates, bisulfates, sulfites, bisulfites, phosphates, monohydrogen-phosphates, dihydrogen phosphates, metaphosphates, pyrophosphates, bromides (such as hydrobromides), chlorides (such as hydrochlorides), iodides (such as hydroiodides), acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

Canagliflozin is commercially available as understood to those skilled in the art. For example, canagliflozin is available as Invokana®. Canagliflozin exhibits inhibitory activity against sodium-dependent glucose transporter, such as for example SGLT2; and may be prepared according to the process as disclosed in US Patent Application Publication No. 2005/0233988, which is incorporated by reference herein.

As used herein, unless otherwise noted, the terms “treating”, “treatment” and the like, shall include the management and care of a patient for the purpose of combating a disease, condition, or disorder. The terms “treating” and “treatment” also include the administration of the compounds or pharmaceutical compositions as described herein to (a) alleviate one or more symptoms or complications of the disease, condition or disorder; (b) prevent the onset of one or more symptoms or complications of the disease, condition or disorder; and/or (c) eliminate one or more symptoms or complications of the disease, condition, or disorder.

As used herein, unless otherwise noted, the terms “preventing”, “prevention” and the like, shall include (a) reducing the frequency of one or more symptoms; (b) reducing the severity of one or more symptoms; (c) delaying, slowing or avoiding of the development of additional symptoms; and/or (d) slowing, or avoiding the development of the disorder or condition to a later stage or more serious form.

One skilled in the art will recognize that, wherein the present disclosure is directed to methods of prevention, a patient in need of thereof shall include any patient or patient who has experienced or exhibited at least one symptom of the disorder, disease or condition to be prevented. Further, a patient in need thereof may additionally be a patient who has not exhibited any symptoms of the disorder, disease or condition to be prevented, but who has been deemed by a physician, clinician or other medical profession to be at risk of developing said disorder, disease or condition. For example, the patient may be deemed at risk of developing a disorder, disease or condition (and therefore in need of prevention or preventive treatment) as a consequence of the patient's medical history, including, but not limited to, family history, pre-disposition, co-existing (comorbid) disorders or conditions, genetic testing, and the like.

The terms “subject” and “patient” are interchangeably used herein to refer to an animal, preferably a mammal, most preferably a human, who has been the object of treatment, observation or experiment. Preferably, the patient has experienced and/or exhibited at least one symptom of the disease or disorder to be treated and/or prevented.

The methods described herein reduce or prevent the incidence of one or more renal events in a patient. Because patients described herein have chronic kidney disease, they are at an increased risk of renal events.

The term “renal event” as used herein refers to disorders related to or affecting kidney function and/or renal hyperfiltration. Renal disorders include, but are not limited to elevated urine albumin level, elevated serum creatinine, elevated serum albumin/creatinine ratio (ACR), renal hyperfiltrative injury, diabetic nephropathy (including, but not limited to hyperfiltrative diabetic nephropathy), renal hyperfiltration, glomerular hyperfiltration, renal allograft hyperfiltration, compensatory hyperfiltration, hyperfiltrative chronic kidney disease, hyperfiltrative acute renal failure, obesity, end-stage kidney disease (ESKD), or renal death.

In some embodiments, the one or more renal events comprises doubling of serum creatinine, end-stage kidney disease, or renal death, or any combination thereof. In other embodiments, the one or more renal events comprises doubling of serum creatinine. In other embodiments, the one or more renal events comprises end-stage kidney disease. In yet further embodiments, the one or more renal events comprises renal death.

The terms “end-stage kidney disease” and “stage 5 kidney disease” are interchangeable and refer to a patient having an eGFR of less than about 15 mL/min/1.73 m². Patients having end-stage kidney disease typically have severe symptoms. Symptoms at this stage may include, without limitation, itching, muscle cramps, nausea, vomiting, no appetite, swelling (often in the hands and feet), pain such as back pain, urinating more or less than normal, difficulties breathing, difficulties sleeping, or any combination thereof. In certain embodiments, patients having end-stage chronic kidney disease, have kidneys that are close to failure or have completely failed. In other embodiments, these patients are on dialysis, require a renal transplant, or any combination thereof.

The methods described herein also reduce or prevent the incidence of one or more cardiovascular events in a patient.

As used herein, unless otherwise noted, the term “cardiovascular event” shall include, but is not limited to, cardiovascular death, non-fatal myocardial infarction, non-fatal stroke (ischemia), peripheral artery disease, hypertensive heart disease, ischemic heart disease, coronary vascular disease, peripheral vascular disease, cerebrovascular disease, cardiac arrhythmia (other than sinus tachycardia), cardiomyopathy, angina (including but not limited to unstable angina), heart failure (including, but not limited to heart failure requiring hospitalization, heart failure, and the like) and coronary valve disease. In some embodiments, the cardiovascular event is cardiovascular death, hospitalized heart failure, non-fatal myocardial infarction, or non-fatal stroke, or any combination thereof. In other embodiments, the cardiovascular event is cardiovascular death, non-fatal myocardial infarction, or non-fatal stroke. In further embodiments, the cardiovascular or disease is non-fatal myocardial infarction. In further embodiments, the cardiovascular event is non-fatal stroke. In yet other embodiments, the cardiovascular event is cardiovascular death. In other embodiments, the cardiovascular event is hospitalized heart failure.

In other embodiments, the patient may also be diagnosed with having one or more cardiovascular risk factor that may lead to a cardiovascular event. The cardiovascular risk factor may include, without limitation, syncope, transient ischemic event or cerebral stroke (except intracranial hemorrhage), cardiovascular surgery such as heart transplant, implantation of a cardiac device such as a cardiac stimulator (pacemaker) or defibrillator (“ICD”), cerebrovascular or peripheral intervention, pulmonary embolism or deep vein thrombosis, acute pulmonary oedema or dyspnoea from cardiac causes, stable angina pectoris or atypical thoracic pain, supraventricular rhythm disorders such as atrial fibrillation, variations in arterial pressure (e.g., hypotension, hypertension, except syncope), cardiovascular infection, major bleeding/hemorrhage (requiring two or more blood cell pellets or any intracranial hemorrhage), elevated cholesterol (hyperlipidemia) such as elevated LDL, depressed HDL, elevated triglycerides, obesity, microalbuminuria, peripheral vascular disease, underlying structural heart disease, atherosclerosis, atrial fibrillation, tachycardia, coronary disease, non-rheumatic heart valve disease, dilated cardiomyopathy of ischemic origin, ablation, supraventricular tachycardia other than atrial fibrillation or flutter, history of heart valve surgery, non-ischemic dilated cardiomyopathy, hypertrophic cardiomyopathy, rheumatic valve disease, sustained ventricular tachycardia, congenital cardiopathy, ventricular fibrillation, at least one cardiac device (including, but not limited to a cardiac stimulator, an implantable defibrillator, and the like), current or past history of smoking, male gender, or any combination thereof. See, e.g., Hohnloser et al., Journal of cardiovascular electrophysiology, January 2008, Vol. 19, No. 1, pages 69-73, which is incorporated herein by reference.

The methods also permit administering a concomitant standard of care. The term “standard of care” typically refers to a physician prescribed treatment of the disease condition at issue. In some embodiments, the standard of care comprises, consists of, or consists essentially of administering an additional pharmaceutical agent that is an angiotensin-converting enzyme inhibitor, angiotensin receptor blocker, or a combination thereof. In some embodiments, the methods include administering an angiotensin-converting enzyme inhibitor. In other embodiments, the methods include administering an angiotensin receptor blocker. In further embodiments, the methods including administering an angiotensin-converting enzyme inhibitor and an angiotensin receptor blocker. Typically, the standard of care does not include treatment by administering canagliflozin. The standard of care may be administered to the patient prior to, subsequently to, or concurrently with canagliflozin. In some embodiments, the standard of care is administered before canagliflozin. In other embodiments, the standard of care is administered after canagliflozin. In further embodiments, the standard of care is administered concurrently with canagliflozin.

As used herein, the terms “angiotensin-converting-enzyme inhibitor,” “ACE inhibitor,” or “ACEi” are interchangeable and refer to a pharmaceutical agent which inhibits the angiotensin-converting enzyme, thereby decreasing the tension of blood vessels and blood volume (i.e., dilating blood vessels), thus lowering blood pressure. ACE inhibitors can be divided into three groups based on their molecular structure: (a) sulfhydryl-containing agents including, but not limited to, alacepril, captopril (CAPOTEN®) and zofenopril; (b) dicarboxylate-containing agents including, but not limited to, enalapril (VASOTEC®), ramipril (ALTACE®, PRILACE®, RAMACE®), quinapril (ACCUPRIL®), perindopril (COVERSYL®, ACEON®), lisinopril (PRINIVIL®, ZESTRIL®), benazepril (LOTENSIN®), imidapril (TANATRIL®, TANAPRESS®, CARDIPRIL®), zofenopril (ZOFECARD®), trandolapril (MAVIK®, ODRIK®), moexipril (UNIVASC®), cilazapril, delapril, spirapril, and temocapril; and (c) phosphonate-containing agents including, but not limited to, fosinopril (FOSITEN®, MONOPRIL®). In some embodiments, the ACE inhibitor is benazepril, captopril, enalapril, imidapril, lisinopril, or ramipril. In other embodiments, the ACE inhibitor is enalapril, imidapril, lisinopril, or ramipril. In further embodiments, the ACE inhibitor is benazepril, captopril, enalapril, imidapril, lisinopril, ramipril, or any combination thereof. One skilled in the art will readily recognize that recommended dosage amounts and regimens for the ACE inhibitors may be determined by consulting appropriate references such as drug package inserts, FDA guidelines, the Physician's Desk Reference, and the like.

As used herein, unless otherwise noted, the terms “ARB,” “angiotensin receptor blocker,” and “angiotensin II receptor antagonist” are interchangeable and refer to a pharmaceutical agent which modulates the renin-angiotensin-aldosterone system. More particularly, ARBs block activation of angiotensin II AT1 receptors, which results in vasodilation (dilation of blood vessels), reduced secretion of vasopressin and reduced production and secretion of aldosterone, among other actions. The combined effect reduces blood pressure. Suitable examples of ARBs include, but are not limited to, losartan (COZAAR®), irbesartan (APROVEL®, KARVEA®, AVAPRO®), olmesartan (BENICAR®), candesartan (BLOPRESS®, ATACAND®), valsartan (DIOVAN®), telmisartan (MICARDIS®), azilsartan (EDARBI®) and eprosartan (TEVETAN®). In some embodiments, the ARB is candesartan, irbesartan, losartan, or valsartan. In other embodiments, the ARB is irbesartan or losartan. One skilled in the art will readily recognize that recommended dosage amounts and regimens for the ARBs may be determined by consulting appropriate references such as drug package inserts, FDA guidelines, the Physician's Desk Reference, and the like.

In yet other embodiments, the incidence of the one or more renal and/or cardiovascular events are reduced or prevented relative to a standard of care comprising administering an angiotensin-converting enzyme inhibitor and/or angiotensin receptor blocker. For example, the methods reduce the risk of incidence and/or the predicted severity of the one or more renal and/or cardiovascular events described herein relative to a subject at the same level of disease progression treated under standard of care, but who is not receiving treatment by administering canagliflozin.

In certain aspects, the methods described herein are effective to reduce the relative risk to a doubling of serum creatinine, ESKD, renal death, or cardiovascular (CV) death, or any combination thereof. In some aspects, the methods reduce the relative risk to a doubling of serum creatinine, ESKD, renal death, or CV death. In other aspects, the methods reduce the relative risk to a doubling of serum creatinine. In further aspects, the methods reduce the relative risk to ESKD. In yet other aspects, the methods reduce the relative risk to renal death. In still further aspects, the methods reduce the relative risk to CV death.

For example, the methods described herein are effective to reduce the risk to a doubling of serum creatinine, ESKD, renal death, or CV death, or any combination thereof, by about, or by at least about, 10%, 12%, 15%, 20%, 25%, 30%, 35%, 40%, 45% or 50%, relative to a patient at the same level of disease progression receiving standard of care, such as a maximum tolerated labeled daily dose of an ACEi and/or ARB, but who is not receiving treatment with canagliflozin. In certain aspects, the reduction is by at least about 25%. In other aspects, the reduction is by at least about 30%. Generally, the reduction in risk to a doubling of serum creatinine, ESKD, renal death, or CV death, or any combination thereof, relative to a patient at the same level of disease progression receiving a standard of care, such as a maximum tolerated labeled daily dose of an ACEi and/or ARB, but who is not receiving treatment with canagliflozin, ranges from about 10% to about 70%, about 10% to about 60, about 10% to about 50%, about 10% to about 40%, about 10% to about 30%, about 10% to about 20%, about 20% to about 70%, about 20% to about 60%, about 20% to about 50%, about 20% to about 40%, about 20% to about 30%, about 30% to about 70%, about 30% to about 60%, about 30% to about 50%, about 30% to about 40%, about 40% to about 70%, about 40% to about 60%, about 40% to about 50%, about 50% to about 70%, about 50% to about 60%, or about 60% to about 70%.

In other embodiments, the methods are effective to reduce the risk to a doubling of serum creatinine, ESKD, or renal death, or any combination thereof, by about, or by at least about, 10%, 12%, 15%, 20%, 25%, 30%, 35%, 40%, 45% or 50%, relative to a patient at the same level of disease progression receiving standard of care, such as a maximum tolerated labeled daily dose of an ACEi and/or ARB, but who is not receiving treatment with canagliflozin. In certain aspects, the reduction is by at least about 25%. In other aspects, the reduction is by at least about 30%. Generally, the reduction in risk to a doubling of serum creatinine, ESKD, or renal death, or any combination thereof, relative to a patient at the same level of disease progression receiving a standard of care, such as a maximum tolerated labeled daily dose of an ACEi and/or ARB, but who is not receiving treatment with canagliflozin, ranges from about 10% to about 70%, about 10% to about 60, about 10% to about 50%, about 10% to about 40%, about 10% to about 30%, about 10% to about 20%, about 20% to about 70%, about 20% to about 60%, about 20% to about 50%, about 20% to about 40%, about 20% to about 30%, about 30% to about 70%, about 30% to about 60%, about 30% to about 50%, about 30% to about 40%, about 40% to about 70%, about 40% to about 60%, about 40% to about 50%, about 50% to about 70%, about 50% to about 60%, or about 60% to about 70%.

In other embodiments, the methods described herein are effective to reduce the risk to CV death, hospitalized heart failure, or a combination thereof, by about, or by at least about, 10%, 12%, 15%, 20%, 25%, 30%, 35%, 40%, 45% or 50%, relative to a patient at the same level of disease progression receiving standard of care, such as a maximum tolerated labeled daily dose of an ACEi and/or ARB, but who is not receiving treatment with canagliflozin. In certain aspects, the reduction is by at least about 25%. In other aspects, the reduction is by at least about 30%. Generally, the reduction in risk to CV death, hospitalized heart failure, or a combination thereof, relative to a patient at the same level of disease progression receiving a standard of care, such as a maximum tolerated labeled daily dose of an ACEi and/or ARB, but who is not receiving treatment with canagliflozin, ranges from about 10% to about 70%, about 10% to about 60, about 10% to about 50%, about 10% to about 40%, about 10% to about 30%, about 10% to about 20%, about 20% to about 70%, about 20% to about 60%, about 20% to about 50%, about 20% to about 40%, about 20% to about 30%, about 30% to about 70%, about 30% to about 60%, about 30% to about 50%, about 30% to about 40%, about 40% to about 70%, about 40% to about 60%, about 40% to about 50%, about 50% to about 70%, about 50% to about 60%, or about 60% to about 70%.

In other embodiments, the methods described herein are effective to reduce the risk to non-fatal MI, non-fatal stroke, or a combination thereof, by about, or by at least about, 10%, 12%, 15%, 20%, 25%, 30%, 35%, 40%, 45% or 50%, relative to a patient at the same level of disease progression receiving standard of care, such as a maximum tolerated labeled daily dose of an ACEi and/or ARB, but who is not receiving treatment with canagliflozin. In certain aspects, the reduction is by at least about 20%. Generally, the reduction in risk to non-fatal MI, non-fatal stroke, or a combination thereof, relative to a patient at the same level of disease progression receiving a standard of care, such as a maximum tolerated labeled daily dose of an ACEi and/or ARB, but who is not receiving treatment with canagliflozin, ranges from about 10% to about 70%, about 10% to about 60, about 10% to about 50%, about 10% to about 40%, about 10% to about 30%, about 10% to about 20%, about 20% to about 70%, about 20% to about 60%, about 20% to about 50%, about 20% to about 40%, about 20% to about 30%, about 30% to about 70%, about 30% to about 60%, about 30% to about 50%, about 30% to about 40%, about 40% to about 70%, about 40% to about 60%, about 40% to about 50%, about 50% to about 70%, about 50% to about 60%, or about 60% to about 70%.

In other embodiments, the methods described herein are effective to reduce the risk to hospitalized heart failure by about, or by at least about, 10%, 12%, 15%, 20%, 25%, 30%, 35%, 40%, 45% or 50%, relative to a patient at the same level of disease progression receiving standard of care, such as a maximum tolerated labeled daily dose of an ACEi and/or ARB, but who is not receiving treatment with canagliflozin. In certain aspects, the reduction is by at least about 35%. Generally, the reduction in risk to hospitalized heart failure, relative to a patient at the same level of disease progression receiving a standard of care, such as a maximum tolerated labeled daily dose of an ACEi and/or ARB, but who is not receiving treatment with canagliflozin, ranges from about 10% to about 70%, about 10% to about 60, about 10% to about 50%, about 10% to about 40%, about 10% to about 30%, about 10% to about 20%, about 20% to about 70%, about 20% to about 60%, about 20% to about 50%, about 20% to about 40%, about 20% to about 30%, about 30% to about 70%, about 30% to about 60%, about 30% to about 50%, about 30% to about 40%, about 40% to about 70%, about 40% to about 60%, about 40% to about 50%, about 50% to about 70%, about 50% to about 60%, or about 60% to about 70%.

In yet further embodiments, the methods described herein are effective to prevent doubling of serum creatinine, end-stage kidney disease (ESKD), renal death, or any combination thereof. In some aspects, the methods are effective to prevent doubling of serum creatinine. In further aspects, the methods are effective to prevent ESKD. In other aspects, the methods are effective to prevent renal death.

The methods described herein, the therapeutically effective amount of canagliflozin is safe, effective, or safe and effective. As used herein, unless otherwise noted, the term “safe” shall mean without undue adverse side effects (such as toxicity, irritation, or allergic response), commensurate with a reasonable benefit/risk ratio when used in the manner of this invention. Similarly, unless otherwise noted, the term “effective” means the efficacy of treatment has been demonstrated for the treatment of patients with chronic kidney disease when dosed in a therapeutically effective dose. In certain embodiments, the methods described herein are safe. In other embodiments, the methods described herein are effective. In further embodiments, the methods described herein are safe and effective. In yet other embodiments, the therapeutically effective amount of canagliflozin is safe. In still further embodiments, the therapeutically effective amount of canagliflozin is effective. In other embodiments, the therapeutically effective amount of canagliflozin is safe and effective.

As used herein, unless otherwise noted, the term “clinically proven” (used independently or to modify the terms “safe” and/or “effective”) shall mean that proof has been proven by a Phase III clinical trial that are sufficient to meet approval standards of U.S. Food and Drug Administration or similar study for market authorization by EMEA. Preferably, an adequately sized, randomized, double-blinded controlled study is used to clinically prove the effects of canagliflozin as compared to a placebo with the patient's condition assessed by techniques described herein.

As used herein, unless otherwise noted, the term “clinically proven effective” means the efficacy of treatment has been proven by a Phase III clinical trial as statistically significant i.e., the results of the clinical trial are not likely to be due to chance with an alpha level less than 0.05 or the clinical efficacy results are sufficient to meet approval standards of U.S. Food and Drug Administration or similar study for market authorization by EMEA. For example, canagliflozin was clinically proven effective for the treatment of patients with chronic kidney disease when dosed in a therapeutically effective dose in reducing the progression of chronic kidney disease as described herein, and as specifically set forth in the examples.

As used herein, unless otherwise noted, the term “clinically proven safe” means the safety of treatment has been proven by a Phase III clinical trial by analysis of the trial data and results establishing that the treatment is without undue adverse side effects and commensurate with the statistically significant clinical benefit (e.g., efficacy) sufficient to meet approval standards of U.S. Food and Drug Administration or similar study for market authorization by Europe, the Middle East, and Africa (EMEA). For example, canagliflozin was clinically proven safe for the treatment of patients with chronic kidney disease when dosed in a therapeutically effective dose as described herein, and as specifically set forth in the examples.

In certain aspects, methods of selling a drug product comprising canagliflozin are also provided. The terms “sale” or “selling” as used herein refers to transferring a drug product, e.g., a pharmaceutical composition or a dosage form, from a seller to a buyer. Thus, the methods include selling a drug product comprising canagliflozin, wherein the method comprises selling the drug product. In some embodiments, a drug product label for a reference listed drug for the drug product includes instructions for treating chronic kidney disease. The methods also include offering for sale a drug product comprising canagliflozin. The term “offering for sale,” as used herein, refers to the proposal of a sale by a seller to a buyer for a drug product, e.g., a pharmaceutical composition or a dosage form. These methods comprise offering the drug product for sale.

The term “drug product” is product that contains an active pharmaceutical ingredient that has been approved for marketing by a governmental authority, e.g., the Food and Drug Administration or the similar authority in other countries. In some embodiments, the drug product comprises canagliflozin.

Similarly, “label” or “drug product label” refers to information provided to a patient which provides relevant information regarding the drug product. Such information includes, without limitation, one or more of the description of the drug, clinical pharmacology, indications (uses for the drug product), contraindication (who should not take the drug product), warnings, precautions, adverse events (side effects), drug abuse and dependence, dosage and administration, use in pregnancy, use in nursing mothers, use in children and older patients, how the drug is supplied, safety information for the patient, or any combination thereof. In certain embodiments, the label or drug product label provides an instruction for use in a patient with Type II diabetes mellitus or macroalbuminuria. In other embodiments, the drug product label comprises data for reducing one or more adverse renal or cardiovascular events relative to a standard of care. In further embodiments, the label or drug product label identifies canagliflozin as a regulatory approved chemical entity. In still other embodiments, the label provides instructions for use in a patient with chronic kidney disease. In yet further embodiments, the label provides a definition of chronic kidney disease and instructs a patient or a physician to administer the canagliflozin if the patient has chronic kidney disease.

The term “reference listed drug” or “RLD” as used herein refers to a drug product to which new generic versions are compared to show that they are bioequivalent. It is also a medicinal product that has been granted marketing authorization by a member state of the European Union or by the Commission on the basis of a completed dossier, i.e., with the submission of quality, pre-clinical and clinical data in accordance with Articles 8(3), 10a, 10b or 10c of Directive 2001/83/EC and to which the application for marketing authorization for a generic/hybrid medicinal product refers, by demonstration of bioequivalence, usually through the submission of the appropriate bioavailability studies.

In the United States, a company seeking approval to market a generic equivalent must refer to the RLD in its Abbreviated New Drug Application (ANDA). For example, an ANDA applicant relies on the FDA's finding that a previously approved drug product, i.e., the RLD, is safe and effective, and must demonstrate, among other things, that the proposed generic drug product is the same as the RLD in certain ways. Specifically, with limited exceptions, a drug product for which an ANDA is submitted must have, among other things, the same active ingredient(s), conditions of use, route of administration, dosage form, strength, and (with certain permissible differences) labeling as the RLD. The RLD is the listed drug to which the ANDA applicant must show its proposed ANDA drug product is the same with respect to active ingredient(s), dosage form, route of administration, strength, labeling and conditions of use, among other characteristics. In the electronic Orange Book, there is a column for RLDs and a column for reference standards. In the printed version of the Orange Book, the RLDs and reference standards are identified by specific symbol.

In Europe, Applicants identify in the application form for its generic/hybrid medicinal product, which is the same as an ANDA or supplemental NDA (sNDA) drug product, the reference medicinal product (product name, strength, pharmaceutical form, marketing authorization holder (MAH, first authorization, Member State/Community), which is synonymous with a RLD, as follows:
1. The medicinal product that is or has been authorized in the European Economic Area (EEA), used as the basis for demonstrating that the data protection period defined in the European pharmaceutical legislation has expired. This reference medicinal product, identified for the purpose of calculating expiry of the period of data protection, may be for a different strength, pharmaceutical form, administration route or presentation than the generic/hybrid medicinal product.
2. The medicinal product, the dossier of which is cross-referred to in the generic/hybrid application (product name, strength, pharmaceutical form, MAH, marketing authorization number). This reference medicinal product may have been authorized through separate procedures and under a different name than the reference medicinal product identified for the purpose of calculating expiry of the period of data protection. The product information of this reference medicinal product will, in principle, serve as the basis for the product information claimed for the generic/hybrid medicinal product.
3. The medicinal product (product name, strength, pharmaceutical form, MAH, Member State of source) used for the bioequivalence study(ies) (where applicable).

The different abbreviated approval pathways for drug products under the Food, Drug, and Cosmetics (FD&C) Act are the abbreviated approval pathways described in sections 505(j) and 505(b)(2) of the FD&C Act (21 U.S.C. 355(j) and 21 U.S.C. 23 355(b)(2), respectively).

According to the FDA (“Determining Whether to Submit an ANDA or a 505(b)(2) Application Guidance for Industry,” U.S. Department of Health and Human Services, October 2017, pp. 1-14, the contents of which is incorporated herein by reference), NDAs and ANDAs can be divided into the following four categories:
(1) A “stand-alone NDA” is an application submitted under section 505(b)(1) and approved under section 505(c) of the FD&C Act that contains full reports of investigations of safety and effectiveness that were conducted by or for the applicant or for which the applicant has a right of reference or use.
(2) A section 505(b)(2) application is an NDA submitted under section 505(b)(1) and approved under section 505(c) of the FD&C Act that contains full reports of investigations of safety and effectiveness, where at least some of the information required for approval comes from studies not conducted by or for the applicant and for which the applicant has not obtained a right of reference or use.
(3) An ANDA is an application for a duplicate of a previously approved drug product that was submitted and approved under section 505(j) of the FD&C Act. An ANDA relies on the FDA's finding that the previously approved drug product, i.e., the reference listed drug (RLD), is safe and effective. An ANDA generally must contain information to show that the proposed generic product (a) is the same as the RLD with respect to the active ingredient(s), conditions of use, route of administration, dosage form, strength, and labeling (with certain permissible differences) and (b) is bioequivalent to the RLD. An ANDA may not be submitted if studies are necessary to establish the safety and effectiveness of the proposed product.
(4) A petitioned ANDA is a type of ANDA for a drug product that differs from the RLD in its dosage form, route of administration, strength, or active ingredient (in a product with more than one active ingredient) and for which FDA has determined, in response to a petition submitted under section 505(j)(2)(C) of the FD&C Act (suitability petition), that studies are not necessary to establish the safety and effectiveness of the proposed drug product.

A scientific premise underlying the Hatch-Waxman Act is that a drug product approved in an ANDA under section 505(j) of the FD&C Act is presumed to be therapeutically equivalent to its RLD. Products classified as therapeutically equivalent can be substituted with the full expectation that the substituted product will produce the same clinical effect and safety profile as the prescribed product when administered to patients under the conditions specified in the labeling. In contrast to an ANDA, a section 505(b)(2) application allows greater flexibility as to the characteristics of the proposed product. A section 505(b)(2) application will not necessarily be rated therapeutically equivalent to the listed drug it references upon approval.

The methods may also comprise, consist of, or consist essentially of placing canagliflozin into the stream of commerce. In certain embodiments, the canagliflozin includes a package insert that contains instructions for safely and effectively treating chronic kidney disease using the canagliflozin.

In further aspects, described herein are methods of selling a pharmaceutical composition containing canagliflozin comprising, consisting of, or consisting essentially of placing the pharmaceutical composition into the stream of commerce. In certain embodiments, the pharmaceutical composition includes a package insert that contains instructions for safely and effectively treating chronic kidney disease using canagliflozin.

In still further aspects, described herein are methods of offering for sale canagliflozin comprising, consisting of, or consisting essentially of offering to place the canagliflozin into the stream of commerce. In certain embodiments, the canagliflozin includes a package insert that contains instructions for safely and effectively treating chronic kidney disease using canagliflozin.

Formulations/Compositions

Pharmaceutical compositions containing canagliflozin as the active ingredient can be prepared by intimately mixing the compound or compounds with a pharmaceutical carrier according to conventional pharmaceutical compounding techniques. As used herein, the terms “composition” and “formulation” are used interchangeably and encompass a product comprising the specified ingredients in the specified amounts, as well as any product, such as a pharmaceutical product, which results, directly or indirectly, from combinations of the specified ingredients in the specified amounts. A summary of pharmaceutical compositions can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

The pharmaceutical compositions or pharmaceutical drug products may be administered by a number of routes as determined by those skilled in the art. Preferably, the pharmaceutical compositions or drug products are administered by route that is suitable for canagliflozin. In some embodiments, the pharmaceutical compositions or drug products are administered orally, parenterally, or any combination thereof. In other embodiments, the pharmaceutical compositions or drug products are administered orally. In further embodiments, the pharmaceutical compositions or drug products are administered parenterally.

The pharmaceutical compositions or pharmaceutical drug products may administered in a form suitable for the selected route of administration. Thus, the pharmaceutical compositions or pharmaceutical drug products may be administered as suspensions, elixirs, solutions, powders, pills such as capsules, tablets, or caplets, pastilles, granules, syrups, thin films, lozenges, sprays, pastes, or injections. In some embodiments, the pharmaceutical compositions or pharmaceutical drug products are administered as injections such as intradermal injections, subcutaneous injections, intramuscular injections, intraosseous injections, intraperitoneal injections, or intravenous injections. In other embodiments, the pharmaceutical compositions or pharmaceutical drug products are administered as suspensions, elixirs, solutions, powders, pills such as capsules (hard or soft), tablets, or caplets, pastilles, granules, syrups, thin films, lozenges, sprays, or pastes. The pills may be formulated for swallowing, chewable, sublingual use, or buccal use, or may be effervescent to be dissolved or dispersed in water prior to administration. In some embodiments, the pharmaceutical product comprises a pill, tablet, powder, sterile parenteral solution, or liquid spray.

The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Thus, for parenteral administration, the pharmaceutical composition or pharmaceutical product is a sterile, parenteral solution. Injectable suspensions or solutions may also be prepared utilizing aqueous carriers along with appropriate additives.

To prepare such pharmaceutical compositions, canagliflozin, as the active ingredient, is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, from about 25 mg to about 500 mg of canagliflozin or any amount or range therein (preferably about 50 mg, about 75 mg, about 100 mg, about 150 mg, about 200 mg, or about 300 mg of canagliflozin. The dosages, however, may be varied depending upon the requirement of the patients, the severity of the condition being treated and the compound being employed. The use of either daily administration or post-periodic dosing may be employed.

Preferably the pharmaceutical compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. For preparing solid compositions such as tablets, the principal active ingredient (e.g., canagliflozin) is mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present disclosure, or a pharmaceutically acceptable salt thereof. In certain embodiments, two active ingredients can be formulated together, e.g., in a bi-layer tablet formulation. When referring to these preformulation compositions as homogeneous, it is meant that the active ingredients are dispersed evenly throughout the composition so that the composition may be readily subdivided into equally effective dosage forms such as tablets, pills and capsules. This solid preformulation composition is then subdivided into unit dosage forms of the type described above containing from about 25 mg to about 500 mg of canagliflozin or any amount or range therein. The tablets or pills of the composition can be coated or otherwise compounded to provide a dosage form affording the advantage of prolonged action. For example, the tablet or pill can comprise an inner dosage and an outer dosage component, the latter being in the form of an envelope over the former.

The liquid forms in which the compositions of the present disclosure may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The methods described herein may also be carried out using a pharmaceutical composition comprising canagliflozin and a pharmaceutically acceptable carrier. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

Advantageously, canagliflozin may be administered in a single daily dose, or the total daily dosage may be administered in divided doses of two, three or four times daily.

For instance, for oral administration in the form of a tablet or capsule, the active drug component (e.g., canagliflozin) can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

To prepare pharmaceutical compositions of the present disclosure, canagliflozin, as the active ingredient, may be intimately admixed with a pharmaceutical carrier according to conventional pharmaceutical compounding techniques, which carrier may take a wide variety of forms depending of the form of preparation desired for administration (e.g., oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain, the disclosure of which is hereby incorporated by reference.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc., the disclosures of which are hereby incorporated by reference.

The present disclosure also provides pharmaceutical products comprising a clinically proven safe and clinically proven effective amount of canagliflozin. Typically, the pharmaceutical product is a package or is packaged.

In some embodiments, the package includes a label. In certain embodiments, the label identifies the canagliflozin as a regulatory approved chemical entity. In other embodiments, the label provides instructions for use in a patient with chronic kidney disease. In further embodiments, the label provides a definition of chronic kidney disease and instructs a patient or a physician to administer the canagliflozin if the patient has chronic kidney disease. In yet other embodiments, the label further comprises an instruction for use in a patient with Type II diabetes mellitus or macroalbuminuria, or both. In still further embodiments, the label includes data indicating a reduction of one or more adverse renal or cardiovascular events relative to a standard of care.

The following Example is provided to illustrate some of the concepts described within this disclosure. While the Example is considered to provide an embodiment, it should not be considered to limit the more general embodiments described herein.

In the following example, efforts have been made to ensure accuracy with respect to numbers used (e.g., amounts, temperature, etc.) but some experimental error and deviation should be accounted for.

### Example 1

Study Design

This was a randomized, double-blind, event-driven, placebo-controlled, parallel-group, 2-arm, multicenter study to evaluate the effects of canagliflozin relative to placebo on progression to doubling of serum creatinine, end-stage kidney disease (ESKD), renal or cardiovascular (CV) death in patients with type 2 diabetes mellitus (T2DM), Stage 2 or 3 chronic kidney disease (CKD) and macroalbuminuria, who were receiving standard of care including a maximum tolerated labeled daily dose of an angiotensin-converting enzyme inhibitor (ACEi) or angiotensin receptor blocker (ARB). The primary objective was to demonstrate the superiority of canagliflozin relative to placebo in reducing the primary composite endpoint. The safety objective was to assess the overall safety and tolerability of canagliflozin.

Randomization included a 1:1 ratio to canagliflozin 100 mg or matching placebo, stratified by screening estimated glomerular filtration rate (eGFR) [≥30 to less than 45, ≥45 to less than 60, ≥60 to less than 90 mL/min/1.73 m²]. The primary efficacy composite endpoint was the time to the first occurrence of doubling of serum creatinine, ESKD, and renal or CV death. The major secondary efficacy endpoints (tested in the following hierarchical order) included:
Composite of CV death and hospitalized heart failure
Composite of CV death, non-fatal MI, and non-fatal stroke (i.e., 3-point “MACE”)
Hospitalized heart failure
Composite of doubling of serum creatinine, ESKD, and renal death
CV death
All-cause death
Composite of CV death, non-fatal MI, non-fatal stroke, hospitalized heart failure, and hospitalized unstable angina

A planned interim analysis was conducted at 413 primary endpoints and the IDMC subsequently recommended to stop the study early for efficacy based on pre-specified stopping criteria.

II. Statistical Methods

The primary analysis was performed in the Intent-To-Treat (ITT) analysis set (i.e., all randomized subjects through end of study) using a stratified Cox proportional hazard model including treatment as the explanatory variable and stratified by screening eGFR. Secondary endpoints were similarly analyzed.

Hypothesis testing for the primary and major secondary efficacy endpoints was performed in a pre-specified hierarchical sequence which proceeded conditionally based on the significance of the prior tests and until an endpoint failed to show significance. As a result of stopping at the interim analysis, the primary endpoint was tested at a two-sided significance level of 0.022 based on the alpha spending function, whereas secondary endpoints were tested at 0.038.

Treatment-emergent safety analyses and summaries were presented using the On-Treatment analysis set (i.e., all treated subjects through 30 days after last dose), whereas analyses of amputation, fracture, and malignancy were performed using the On-Study analysis set (i.e., all treated subjects through end of study).

III. Results

1. Subject and Treatment Information

1.1. Study Completion/Withdrawal Information

A total of 4,401 randomized subjects were included in the ITT analysis set, and as only 4 subjects were not dosed, a total of 4397 subjects were included in both the On-Study and On-Treatment analysis sets for safety evaluation. Known final vital status and study completion were both very high. With respect to Table 1, a subject is considered as having completed the study, regardless of whether the subject is on or off study drug, if the subject is followed until a time point between the notification of the global trial end date (GTED) and the GTED, or until the time of death for subjects who died prior to the GTED. A greater proportion of subjects discontinued in the placebo group as compared to the canagliflozin group, with an adverse event cited as the most frequent reason for treatment discontinuation (13% in the placebo group and 12% in the canagliflozin group, respectively).

TABLE 1
Study Completion/Withdrawal Information

| | Placebo | Canagliflozin | Total |
|---|---|---|---|
| | (N = 2199) | (N = 2202) | (N = 4401) |
| | n (%) | n (%) | n (%) |
| Subjects in ITT analysis set | 2199 (100) | 2202 (100) | 4401 (100) |
| Completed Study* | 2174 (98.9) | 2187 (99.3) | 4361 (99.1) |
| Final vital status known** | 2197 (99.9) | 2198 (99.8) | 4395 (99.9) |
| Alive | 1995 (90.7) | 2030 (92.2) | 4025 (91.5) |
| Died | 202 (9.2) | 168 (7.6) | 370 (8.4) |
| Final Vital Status Unknown | 2 (0.1) | 4 (0.2) | 6 (0.1) |

*Not including results from the search of public records.
**Including results from the search of public records.

1.2. Demographic and Baseline Characteristics

Both final vital status (99.9%) and study completion (99.1%) were very high. The most frequent reason cited for early discontinuation from study drug was an adverse event (12% in the canagliflozin group and 13% in the placebo group). There were no notable differences in the baseline demographic, anthropometric, and diabetic characteristics between the two treatment groups. Overall, the mean age was 63 years; 66.1% of subjects were male, and the majority were white (66.6%). The mean duration of diabetes was 16 years, mean baseline HbA_{1c} was 8.27%, with 53.2% of subjects having baseline HbA_{1c}≥8% (mean HbA_{1c} of 8.27%), and baseline median urine albumin/creatinine was 927 mg/g. The most frequent antihyperglycemic agents (AHA) medications used at baseline were insulin (65.5%), biguanides (57.8%), and sulfonylureas (28.8%). Nearly all subjects (99.9%) were on ACEi or ARB at randomization, and 95% were on an ACEi or ARB at 2 years after randomization. About 92% of the subjects were on cardiovascular therapies (not including ACEi/ARBs) at baseline, with approximately 60% taking an anti-thrombotic agent (including aspirin) and 69% on statins/hypertensive drugs.

The mean baseline eGFR was 56.2 mL/min/1.73 m² and approximately 60% of the population had a baseline eGFR of less than 60 mL/min/1.73 m². Subjects had a mean duration of diabetes of approximately 16 years. The proportion of subjects with prior CV disease was 50.4%; 14.8% had a history of heart failure; 5.3% had a history of amputation. While the entire study population had nephropathy at baseline, about 64% of the population had at least 2 microvascular complications (i.e., diabetic nephropathy and another microvascular complication). No clinically relevant differences among treatment groups in these baseline characteristics were noted.

The proportion of subjects with a history of amputation was similar between groups (5.4% in the canagliflozin group and 5.2% in the placebo group).

1.3. Extent of Exposure and Follow-up

The mean exposure to study drug was comparable between the treatment groups (115 weeks overall) as was the duration of follow-up (136 weeks overall).

2. Primary Endpoint Analysis

Canagliflozin significantly reduced the risk of the primary composite endpoint as compared to placebo by 30% [HR: 0.70, 95% CI: 0.59, 0.82, p-value <0.0001] (Table 2A), thereby successfully meeting the primary objective of the study. Additionally, each individual component was consistent with the overall results of the primary composite endpoint (Table 2A) and furthermore, a supportive analysis using the On-Treatment analysis set [HR: 0.64, 95% CI: 0.53, 0.78] was consistent with the primary efficacy analysis in the ITT analysis set.

TABLE 2A
Analysis of the Primary Composite Endpoint (Including the Individual Components)

| | | | | | Canagliflozin | |
|---|---|---|---|---|---|---|
| | | Placebo | | | HR ^{b} | |
| Endpoint | n/N (%) | EVRT ^{a} | n/N (%) | EVRT ^{a} | (95% CI) | P-value ^{b} |
| Primary Composite | 340/2199 (15.5) | 61.24 | 245/2202 (11.1) | 43.21 | 0.70 (0.59, 0.82) | <0.0001 |
| Endpoint | | | | | | |
| Doubling of Serum | 188/2199 (8.5) | 33.78 | 118/2202 (5.4) | 20.73 | 0.60 (0.48, 0.76) | <0.0001 |
| Creatinine | | | | | | |
| ESKD | 165/2199 (7.5) | 29.44 | 116/2202 (5.3) | 20.37 | 0.68 (0.54, 0.86) | 0.0015 |
| Renal Death* | 5/2199 (0.2) | 0.87 | 2/2202 (0.1) | 0.35 | | |
| CV Death | 140/2199 (6.4) | 24.38 | 110/2202 (5.0) | 19.01 | 0.78 (0.61, 1.00) | 0.0502 |

*HR is not presented for renal death due to the small number of events in each group.
^{a} Event rate per 1000 patient-years.
^{b} Hazard ratio (canagliflozin compared to placebo), 95% CI and p-value are estimated using a stratified Cox proportional hazards model including treatment as the explanatory variable and stratified by screening eGFR (≥30 to <45, ≥45 to <60, ≥60 to <90 mL/min/1.73 m²).

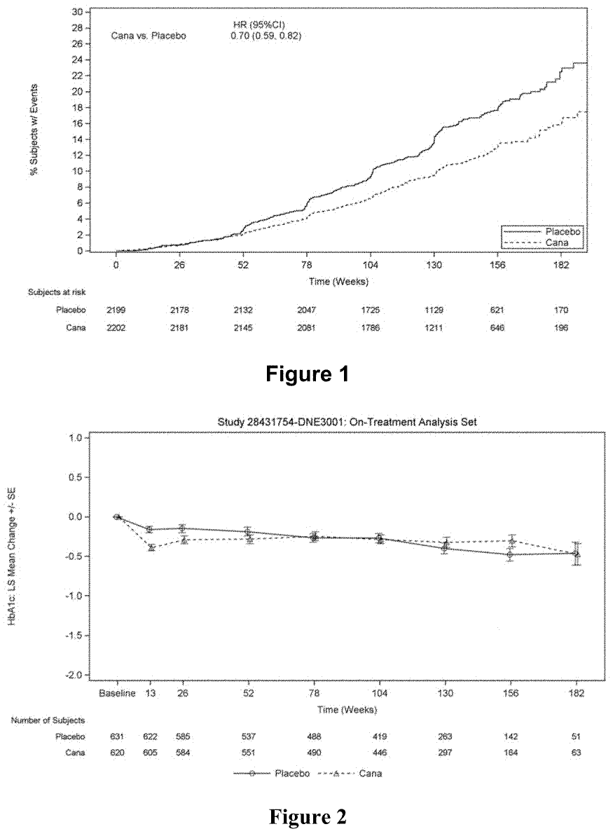

FIG. 1 depicts the Kaplan-Meier plot for the time to first occurrence of the primary composite endpoint and shows an initial separation occurring at Week 52 and was maintained across the entire study. The robustness of the primary composite endpoint, as shown in Tables 2B and 2C, was demonstrated across all 15 subgroups. An assessment of the proportional hazards assumption upon which the above analysis was based yielded no evidence for lack of proportionality (p=0.3116).

3. Major Secondary Endpoints

Canagliflozin significantly reduced the risk of the following secondary endpoints (shown with bolded p-values in Table 3B):
Composite of CV death and hospitalized heart failure by 31% [HR: 0.69; p=0.0001; 95% CI: 0.57, 0.83]
MACE by 20% [HR: 0.80; p=0.0121; 95% CI: 0.67, 0.95]
Hospitalization for heart failure by 39% [HR: 0.61; p=0.0003; 95% CI: 0.47, 0.80]
Composite of doubling of serum creatinine, ESKD, and renal death by 34% [HR: 0.66; p<0.000195% CI: 0.53, 0.81].

While the remaining secondary endpoints trended toward favoring canagliflozin, due to the hierarchical testing sequence, none were statistically significant. Additionally, as canagliflozin reduced the risk of the exploratory hard composite of ESKD, renal death and CV death [HR: 0.73; 95% CI: 0.61, 0.87], the treatment effect remained consistent regardless of whether the component of doubling of serum creatinine was included. With respect to Tables 3A and 3B, HHF=Hospitalized Heart Failure; DoSC=Doubling of Serum Creatinine. NFMI=Non-Fatal MI; NF Stroke=Non-Fatal Stroke; HUSA=Hospitalized Unstable Angina.

TABLE 3A
Analysis of Secondary Endpoints

| | Placebo | | Canagliflozin | |
|---|---|---|---|---|
| Endpoint | n/N (%) | EVRT ^{a} | n/N (%) | EVRT ^{a} |
| Composite of CV Death/ | 253/2199 | 45.44 | 179/2202 | 31.47 |
| HHF | (11.5) | | (8.1) | |
| MACE | 269/2199 | 48.67 | 217/2202 | 38.71 |
| | (12.2) | | (9.9) | |
| HHF | 141/2199 | 25.33 | 89/2202 | 15.65 |
| | (6.4) | | (4.0) | |
| Composite of DoSC/ | 224/2199 | 40.36 | 153/2202 | 26.99 |
| ESKD/Renal Death | (10.2) | | (6.9) | |
| CV Death | 140/2199 | 24.38 | 110/2202 | 19.01 |
| | (6.4) | | (5.0) | |
| All-Cause Mortality | 201/2199 | 35.00 | 168/2202 | 29.04 |
| | (9.1) | | (7.6) | |
| Composite of CV Death/ | 361/2199 | 66.95 | 273/2202 | 49.35 |
| NFMI/NF Stroke/ | (16.4) | | (12.4) | |
| HHF/HUSA | | | | |

^{a} Event rate per 1000 patient-years.

4. Other Efficacy Analyses

At the end of treatment, statistically significant placebo-subtracted reductions from baseline (canagliflozin minus placebo) were observed for subjects treated with canagliflozin with respect to HbA_{1c} [least-squares mean difference: −0.13%], body weight [least-squares mean difference: −1.72%] and systolic blood pressure [least-squares mean difference: −2.81 mmHg]. The difference in systolic blood pressure noted between treatment groups was unlikely to have had a significant impact on the main efficacy findings, given that subjects were already reasonably well controlled with a mean baseline systolic blood pressure of 140 mmHg (Home, Impact of the UKPDS: an overview, Diabet. Med., 2008, 25:2-8; Kazama, Chronic kidney disease and fragility fracture, Clin. Exp. Nephrol., 2017, 21(Suppl 1):S46-S52; Zoungas, Combined effects of routine blood pressure lowering and intensive glucose control on macrovascular and microvascular outcomes in patients with type 2 diabetes: New results from the ADVANCE trial, 2009, Diabetes Care 32:2068-2074).

5. Safety

Safety analyses were performed using either the On-Treatment analysis set (treated subjects up to 30 days of last dose) or the On-Study analysis set (treated subjects through the end of study). As discussed herein, the incidence of amputations [HR 1.11; 95% CI: 0.79 to 1.56] and adjudicated fractures [HR 0.98; 95% CI: 0.70 to 1.37] were lower in this study versus other studies in the art. The incidence rate of adverse events overall, as well as serious adverse events, were numerically lower in the canagliflozin group compared to placebo.

In summary, the incidence rate of adverse events overall, as well as serious adverse events, and adverse events leading to discontinuation were numerically lower in the canagliflozin group compared to placebo. There was no increase in the rate of adverse events of hyperkalemia, and a numerical increase in the rate of adverse event related to volume depletion.

5.1. Summary of All Adverse Events

The incidence rate of adverse events overall, as well as serious adverse events, and adverse events leading to discontinuation were numerically lower in the canagliflozin group compared to placebo (Table 4).

TABLE 4
Summary of Any Adverse Events - Exposure adjusted

| | Placebo | | Canagliflozin | |
|---|---|---|---|---|
| | (N = 2197) | | (N = 2200) | |
| | | Rate/ | | Rate/ |
| | | 1000 | | 1000 |
| | N (%) | pt-yrs** | N (%) | pt-yrs** |
| Any Adverse Events | 1860 (84.7) | 379.28 | 1784 (81.1) | 351.40 |
| Adverse Events Leading to | 286 (13.0) | 58.32 | 267 (12.1) | 52.59 |
| Discontinuation | | | | |
| Adverse Events Related to | 361 (16.4) | 73.61 | 469 (21.3) | 92.38 |
| Study Drug* | | | | |
| Adverse Events Related to | 55 (2.5) | 11.22 | 75 (3.4) | 14.77 |
| Study Drug* and Leading | | | | |
| to Discontinuation | | | | |
| Serious Adverse Events | 806 (36.7) | 164.36 | 737 (33.5) | 145.17 |
| Serious Adverse Events | 159 (7.2) | 32.42 | 134 (6.1) | 26.39 |
| Leading to Discontinuation | | | | |
| Serious Adverse Events | 42 (1.9) | 8.56 | 62 (2.8) | 12.21 |
| Related to Study Drug* | | | | |
| Serious Adverse Events | 15 (0.7) | 3.06 | 16 (0.7) | 3.15 |
| Related to Study Drug* | | | | |
| and Leading to | | | | |
| Discontinuation | | | | |
| Death*** | 122 (5.6) | 24.88 | 109 (5.0) | 21.47 |

Note:
Percentages calculated with the number of subjects in each group as the denominator.
*Related to study drug includes relationship determined by investigators: possibly related, probably related and very likely related.
**The Denominator is the total of each subject's exposure of the study medication plus 30 days.
***Death is based on the number of subjects who experienced a fatal adverse event.

5.2. Selected Adverse Events of Interest

5.2.1. Lower Limb Amputation

Despite the relatively high historical incidence rate of atraumatic lower extremity amputation in this population, there was no statistical difference in the risk of atraumatic lower limb amputation between treatment groups, given the inclusion of ‘1.00’ in the 95% confidence interval for the hazard ratio comparing canagliflozin to placebo [HR: 1.11; 95% CI: 0.79, 1.56] (Table 5).

TABLE 5
Hazard Ratio of Atraumatic Lower Limb Amputation

| | Placebo | Canagliflozin | |
|---|---|---|---|
| | (N = 2197) | (N = 2200) | |
| Subjects with any event, n (%) | 63 (2.9) | 70 | (3.2) |
| Number of events | 96 | 87 | |
| Subject-year of exposure to first event | 5632 | 5672 | |
| Incidence rate (/1000 subject-years) ^{a} | 11.19 | 12.34 | |
| IRD (/1000 subject-years) (minus | | 1.16 | (−2.87, 5.18) |
| Placebo) ^{b} | | | |
| HR (vs. Placebo) ^{c} | | 1.11 | (0.79, 1.56) |

^{a} Incidence is based on the number of subjects with at least one amputation and not number of events.
^{b} 95% CI is based on Normal approximation for the incidence rate difference (IRD).
^{c} Hazard ratio (HR) is from a Cox proportional hazards model.

5.2.2. Fracture

There was no statistical difference in the risk of adjudicated fracture between treatment groups with incidence rates of 12.09 and 11.80 per 1000 subjects-years in the placebo and canagliflozin groups, respectively, and an estimated hazard ratio close to 1.00 [HR: 0.98; 95% CI: 0.70, 1.37] (Table 6).

TABLE 6
Hazard Ratio of Adjudicated Fracture

| | Placebo | Canagliflozin | |
|---|---|---|---|
| | (N = 2197) | (N = 2200) | |
| Subjects with any event, n (%) | 68 (3.1) | 67 | (3.0) |
| Number of events | 79 | 80 | |
| Subject-year of exposure to first event | 5624 | 5678 | |
| Incidence rate(/1000 subject-years) ^{a} | 12.09 | 11.80 | |
| IRD(/1000 subject-years) (minus | | −0.29 | (−4.35, 3.77) |
| Placebo) ^{b} | | | |
| HR (vs. Placebo) ^{c} | | 0.98 | (0.70, 1.37) |

^{a} Incidence is based on the number of subjects with at least one amputation and not number of events.
^{b} 95% CI is based on Normal approximation for the incidence rate difference (IRD).
^{c} Hazard ratio (HR) is from a Cox proportional hazards model.

5.2.3. Selected Malignancies

The overall incidence rate of neoplasms was low and balanced between the treatment groups.

5.2.3.1. Renal Cell Carcinoma

The incidence of adjudicated renal cell carcinoma (RCC) was numerically higher in the placebo group [5 subjects (0.2%)] as compared to the canagliflozin group [1 subject (<0.1%)]. Specifically, the proportion of subjects who experienced confirmed RCC was low, and there was a numerical decrease in the rate of adjudicated RCC in subjects treated with canagliflozin relative to placebo (incidence rate 0.87 and 0.17 per 1000 patient-years in the placebo and canagliflozin groups, respectively).

5.2.3.2. Other Malignancies

The incidence rate of bladder cancer per 1,000 subject-years was similar in both treatment groups [incidence rate difference (IRD): 0.16; 95% CI: −1.41, 1.73] whereas the incidence rate for female breast cancer was numerically higher in the canagliflozin group as compared to placebo (incidence rate of 1.59 and 4.08 per 1000 patient-years in the placebo and canagliflozin groups, respectively), however the 95% confidence interval for the IRD contained ‘0’ [IRD: 2.49; 95% CI: −1.25, 6.23]. There were no reports of pheochromocytoma or testicular cell cancer reported in the study. With respect to Table 7, percentages are calculated with the number of subjects in each group as denominator, incidence is based on the number of subjects experiencing at least one adverse event, not the number of events, 95% CI is based on the Normal approximation for the incidence rate difference (IRD), and denominators are restricted to females for breast cancer.

TABLE 7
Rate Difference and 95% CI of Selected Post-Randomization Malignancies

| | Placebo | | Canagliflozin | | Canagliflozin | |
|---|---|---|---|---|---|---|
| | (N = 2197) | | (N = 2200) | | vs. Placebo | |
| | | Rate (/1000 | | Rate (/1000 | IRD (/1000 | |
| | n (%) | subject years) | n (%) | subject years) | subject years) | 95% CI |
| Malignancy (Bladder) | 9 (0.4) | 1.57 | 10 (0.5) | 1.73 | 0.16 | (−1.41, 1.73) |
| Malignancy (Breast) | 3 (0.4) | 1.59 | 8 (1.1) | 4.08 | 2.49 | (−1.25, 6.23) |

5.2.4. Renal-Related Adverse Events

The incidence rate for renal-related adverse events was lower in the canagliflozin group compared to placebo (57.12 versus 79.12 per 1,000 subject-years, respectively). The most frequently reported preferred term for both groups was “Blood Creatinine increased.” This finding is reassuring in this population at particular risk for renal adverse events, and further strengthens the role of canagliflozin in reducing the risk of renal outcomes in the population studied. With respect to Table 8, the percentages are calculated with the number of subjects in each group as the denominator and incidence is based on the number of subjects experiencing at least one adverse event, not the number of events.

TABLE 8
Renal Related Adverse Events by Preferred Term

| | Placebo | | Canagliflozin | |
|---|---|---|---|---|
| | (N = 2197) | | (N = 2200) | |
| Dictionary-Derived Term | n (%) | | n (%) | |
| Total no. subjects with the AEs | 388 | (17.7) | 290 | (13.2) |
| Incidence rate per 1000 person-years | 79.12 | | 57.12 | |
| Acute kidney injury | 98 | (4.5) | 86 | (3.9) |
| Anuria | 0 | | 1 | (<0.1) |
| Azotemia | 4 | (0.2) | 0 | |
| Blood Creatinine increased | 203 | (9.2) | 144 | (6.5) |
| Blood urea increased | 21 | (1.0) | 21 | (1.0) |
| Glomerular filtration rate decreased | 81 | (3.7) | 68 | (3.1) |
| Nephropathy toxic | 2 | (0.1) | 0 | |
| Renal failure | 17 | (0.8) | 10 | (0.5) |
| Renal impairment | 68 | (3.1) | 50 | (2.3) |

5.2.5. Other Selected Adverse Events

The incidence rate of hyperkalemia adverse events was lower in the canagliflozin group compared to placebo (29.74 versus 36.91 per 1,000 subject-years, respectively) whereas the incidence rate of volume depletion adverse events was higher in the canagliflozin group compared to placebo (28.36 versus 23.45 per 1,000 subject-years, respectively). The incidence rate of adjudicated diabetic ketoacidosis (DKA) per 1,000 subject-years was higher in the canagliflozin group compared to placebo, with the 95% confidence interval for the IRD excluding ‘0’ [IRD: 1.73; 95% CI: 0.32, 3.14].

6. Other Effects

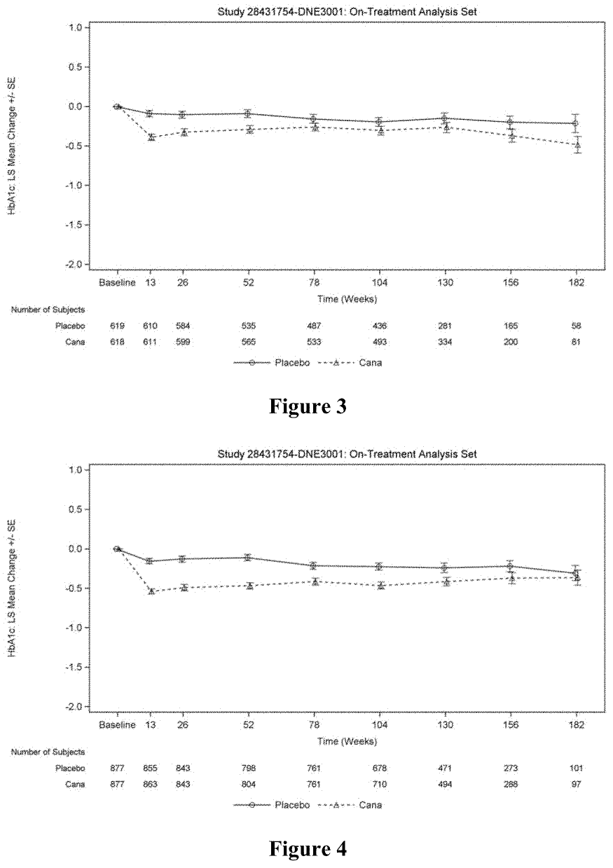

Although statistically significant placebo-subtracted reductions from baseline were observed for subjects treated with canagliflozin with respect to HbA_{1c}, body weight, and systolic blood pressure, the treatment effects were small. Effects on glycemia decreased with lower eGFRs. By eGFR stratum the placebo-subtracted difference of LS means for change from baseline for HbA_{1c} were 0.03 (95% CI: −0.128; 0.180) in the 30-45 eGFR stratum, −0.18 (−0.328; −0.030) in the 45-60 stratum, and −0.21 (95% CI: −0.33; −0.08) in the 60 to <90 eGFR stratum. See, FIGS. 2-4 which show the LS mean change from baseline in A_{1c} over time for each of the eGFR strata. Similar trends were observed with the effects on body weight and systolic blood pressure.

Analyses were performed to assess the impact of adjusting for postbaseline measurements of HbA_{1c} and systolic blood pressure in the primary efficacy analysis. A series of proportional hazard regression models which included postbaseline HbA_{1c} and systolic blood pressure measurements as time-varying covariates were fit. Because changes in HbA₁_{_{c}} have a delayed effect on cardiovascular risk, several models were constructed. In each model, HbA_{1c} was evaluated first using the single coincident value, a single lagged value, and then as a running mean average value. Single coincident systolic blood pressure measurements were used for all analyses.

As shown in Table 9, the treatment effect of canagliflozin on the primary composite endpoint remains robust regardless of the approach for adjusting for time-varying HbA_{1c} and systolic blood pressure measurements. While coincident HbA_{1c} demonstrated no significant effect, both the lagged value and running mean HbA_{1c} showed a modest but significant effect on the primary efficacy endpoint, as did coincident systolic blood pressure. Both higher postbaseline HbA_{1c} and systolic blood pressure were associated with an increased risk of experiencing the primary composite endpoint, after adjustment for the effect of treatment.

TABLE 9
Effects of Time-Varying Risk Factors HbA_{1c} and Systolic Blood Pressure
on the Hazard Ratio for the Primary Efficacy Outcome

| Representation of Time-Varying | | | |
|---|---|---|---|
| HbA_{1c} in the Cox Model | Parameter | HR | P-value |
| Concurrent | Treatment | 0.724 | 0.0001 |
| | HbA_{1c} (%) | 1.021 | 0.4659 |
| | Systolic Blood | 1.013 | <0.0001 |
| | Pressure (mmHg) | | |
| Lagged By 1 Visit | Treatment | 0.737 | 0.0003 |
| | HbA_{1c} (%) | 1.126 | <0.0001 |
| | Systolic Blood | 1.013 | <0.0001 |
| | Pressure (mmHg) | | |
| Running Mean | Treatment | 0.740 | 0.0004 |
| | HbA_{1c} (%) | 1.146 | <0.0001 |
| | Systolic Blood | 1.013 | <0.0001 |
| | Pressure (mmHg) | | |

Note:
A Cox Model is fit to the primary efficacy outcome, stratified by screening eGFR strata (≥30 to <45, ≥45 to <60, ≥60 to <90 mL/min/1.73 m²), with randomized treatment as a fixed effect over time and with systolic blood pressure and HbA_{1c} as time-varying covariates.
Note:
Systolic blood pressure is represented by its measured value at each visit. The representation of HbA_{1c} varies between the models:
Concurrent - HbA_{1c} takes its measured value at each visit
Lagged By 1 Visit - HbA_{1c} takes its measured value at the prior visit
Running Mean - HbA_{1c} takes the time-weighted mean of its measured values over all visits from Baseline to each visit

These results demonstrated that treatment with canagliflozin was able to achieve favorable effects on renal function, ESKD and CV or renal death with only modest treatment-related differences in glycemic control. In general, treatment with canagliflozin demonstrated an improvement in renal outcomes in patients receiving standard of care treatments for diabetes and CV and renal event prevention (which included the maximal labeled or tolerated dose of an ARB or ACE inhibitor), without meaningful differences in achieved glycemic, blood pressure, or lipid control.

7. Summary of Results

In conclusion, this example illustrates a significant advancement in the management of subjects with T2DM and established CKD. Despite the current standard of care, which was established over 15 years ago, subjects with T2DM and CKD are at high risk of developing ESKD and CV events, and of having a reduced life expectancy. Canagliflozin has been shown to significantly reduce the risk of clinically important renal and CV events for these subjects, with an acceptable safety profile.

It is to be understood that while the invention has been described in conjunction with the preferred specific embodiments thereof, that the foregoing description and the examples that follow are intended to illustrate and not limit the scope of the invention. It will be understood by those skilled in the art that various changes may be made and equivalents may be substituted without departing from the scope of the invention, and further that other aspects, advantages and modifications will be apparent to those skilled in the art to which the invention pertains. In addition to the embodiments described herein, the present invention contemplates and claims those inventions resulting from the combination of features of the invention cited herein and those of the cited prior art references which complement the features of the present invention. Similarly, it will be appreciated that any described material, feature, or article may be used in combination with any other material, feature, or article, and such combinations are considered within the scope of this invention.

The disclosures of each patent, patent application, and publication cited or described in this document are hereby incorporated herein by reference, each in its entirety, for all purposes.

1. A method of treating a diabetic patient with chronic kidney disease, comprising:
(a) determining whether the patient has chronic kidney disease; and
(b) administering canagliflozin to the patient in a therapeutically effective amount to treat the chronic kidney disease.

2. The method of claim 1, wherein the patient is a human.

3. The method of claim 1, wherein the diabetes is Type II diabetes mellitus.

4. The method of claim 1, wherein the chronic kidney disease is determined by a blood test, urine test, kidney imaging, or kidney biopsy.

5. The method of claim 1, wherein the chronic kidney disease is determined by estimated glomerular filtration rate.

6. The method of claim 1, wherein the patient has a measured HbA_{1c} in the range of ≥7.0% and ≤10.5%.

7. The method of claim 1, wherein the chronic kidney disease is stage 2 and/or stage 3 chronic kidney disease.

8. The method of claim 1, wherein the patient has an estimated glomerular filtration rate (eGFR) of ≥30 to <90 mL/min/1.73 m².

9. The method of claim 4, wherein the patient has an eGFR of ≥30 to <45 mL/min/1.73 m².

10. The method of claim 4, wherein the patient has an eGFR of ≥45 to <60 mL/min/1.73 m².

11. The method of claim 4, wherein the patient has an eGFR of ≥60 to <90 mL/min/1.73 m².

12. The method of claim 1, wherein the patient has macroalbuminuria.

13. The method of claim 1, wherein the method prevents doubling of serum creatinine, end-stage kidney disease (ESKD), renal death, or any combination thereof.

14. The method of claim 1, wherein the method prevents cardiovascular death, hospitalized heart failure, non-fatal myocardial infarction, non-fatal stroke, or any combination thereof.

15. The method of claim 1, further comprising a concomitant standard of care.

16. The method of claim 15, wherein the standard of care comprises administering an angiotensin-converting enzyme inhibitor and/or angiotensin receptor blocker.

17. The method of claim 1, wherein the risk to a doubling of serum creatinine, ESKD, renal death, or cardiovascular death is reduced by at least about 25% relative to a patient at the same level of disease progression receiving a standard of care, the standard of care comprising administering an angiotensin-converting enzyme inhibitor and/or angiotensin receptor blocker, but not including treatment with canagliflozin.

18. The method of claim 1, wherein the risk to cardiovascular death or hospitalized heart failure is reduced by at least about 25% relative to a patient at the same level of disease progression receiving a standard of care, the standard of care comprising administering an angiotensin-converting enzyme inhibitor and/or angiotensin receptor blocker, but not including treatment with canagliflozin.

19. The method of claim 1, wherein the risk to non-fatal MI or non-fatal stroke is reduced by at least about 20% relative to a patient at the same level of disease progression receiving a standard of care, the standard of care comprising administering an angiotensin-converting enzyme inhibitor and/or angiotensin receptor blocker, but not including treatment with canagliflozin.

20. The method of claim 1, wherein the risk to hospitalized heart failure is reduced by at least about 35% relative to a patient at the same level of disease progression receiving a standard of care, the standard of care comprising administering an angiotensin-converting enzyme inhibitor and/or angiotensin receptor blocker, but not including treatment with canagliflozin.

21. The method of claim 1, wherein the therapeutically effective amount of canagliflozin is about 50 to about 500 mg.

22. The method of claim 21, wherein the therapeutically effective amount of canagliflozin is about 100 to about 300 mg.

23. The method of claim 22, wherein the therapeutically effective amount of canagliflozin is about 100 mg.

24. A method for treating chronic kidney disease, comprising administering to a patient in need thereof, a therapeutically effective amount of canagliflozin, wherein the patient is diagnosed with Type II diabetes mellitus.

25. The method of claim 24, wherein the patient has a measured HbA_{1c} in the range of ≥7.0% and ≤10.5%.

26. The method of claim 24, wherein the chronic kidney disease is stage 2 and/or stage 3 chronic kidney disease.

27. The method of claim 24, wherein the patient has an estimated glomerular filtration rate (eGFR) of ≥30 to <90 mL/min/1.73 m².

28. The method of claim 27, wherein the patient has an eGFR of ≥30 to <45 ml/min/1.73 m².

29. The method of claim 27, wherein the patient has an eGFR of ≥45 to <60 mL/min/1.73 m².

30. The method of claim 27, wherein the patient has an eGFR of ≥60 to <90 mL/min/1.73 m².

31. The method of claim 24, wherein the patient is further diagnosed with macroalbuminuria.

32. The method of claim 24, wherein incidence of one or more renal events is reduced or prevented.

33. The method of claim 32, wherein the one or more renal events comprise doubling of serum creatinine, end-stage kidney disease (ESKD), renal death, or any combination thereof.

34. The method of claim 24, wherein incidence of one or more cardiovascular events is reduced or prevented.

35. The method of claim 34, wherein the one or more cardiovascular events comprise cardiovascular death, hospitalized heart failure, non-fatal myocardial infarction, non-fatal stroke, or any combination thereof.

36. The method of claim 35, wherein the one or more cardiovascular events comprise cardiovascular death, non-fatal myocardial infarction, or non-fatal stroke.

37. The method of claim 24, further comprising a concomitant standard of care comprising administration of an angiotensin-converting enzyme inhibitor and/or angiotensin receptor blocker.

38. The method of claim 24, wherein the incidence of the one or more renal and/or cardiovascular events are reduced or prevented relative to a standard of care, the standard of care comprising administering an angiotensin-converting enzyme inhibitor and/or angiotensin receptor blocker, but not including treatment with canagliflozin.

39. The method of claim 34, wherein the therapeutically effective amount of canagliflozin is about 50 to about 500 mg.

40. The method of claim 39, wherein the therapeutically effective amount of canagliflozin is about 100 to about 300 mg.

41. The method of claim 39, wherein the therapeutically effective amount of canagliflozin is about 100 mg.

42.-53. (canceled)
